(19) United States
(12) Patent Application Publication
(10) Pub. No.: US 2008/0058272 A1
(43) Pub. Date: Mar. 6, 2008

(54) Nonamer Peptides for Cancer Treatment

(76) Inventors: Juergen Becker, Wuerzberg (DE); Valeska Hofmeister, Wuerzberg (DE); David Schrama, Hoechberg (DE)

Correspondence Address: SUZANNAH K. SUNDBY;SMITH, GAMBRELL & RUSSEL, LLP, 1130 Connecticut Avenue, NW, Suite 1130, WASHINGTON, DC, 20036, US

(21) Appl. No.: 11/467,933

(22) Filed: Aug. 29, 2006

(51) Int. Cl. A61K 38/10; C07K 7/08

(52) U.S. Cl. 514/15; 530/328

(57) ABSTRACT

The present invention provides nonamer peptides derived from fibroblast activation protein α (FAPα) for the treatment of solid tumors. These peptides specifically bind to HLA, defined by an IC₅₀ value of less than about 50 μM, induce a T cell response in a subject, wherein position No. 2 of said nonamer peptide is leucine (L), isoleucine (I) or methionine (M), and position No. 9 of said nonamer peptide is leucine (L), valine (V) or isoleucine (I). Further, a composition comprising a nonamer peptide and methods for the prophylactic and therapeutic treatment are provided.

### FIELD OF THE INVENTION

The present invention relates to the technical field of prophylactic and therapeutic cancer treatment of solid tumors. In particular, the present invention relates to nonamer peptides derived from fibroblast activation protein α (FAPα).

### BACKGROUND OF THE INVENTION

It is estimated that in the U.S. about 1,399,790 cancer cases would be diagnosed and 564,830 individuals would die from cancer in 2006 (U.S. Cancer Statistics Working Group 2006). While early, localized disease may effectively be treated by complete excision; metastatic cancer in most cases is fatal. Nevertheless, some patients show spontaneous regression of both primary tumors and metastases. This event is largely attributed to adaptive immune responses, and the presence of cytoxic T cells (CTLs) infiltrating the tumor is associated with a better clinical prognosis (Cho et al., 2003). In addition, the increased tumor incidence in immune suppressed individuals indicates that cancer, at least in part, can be controlled by the immune system (Adami et al., 2003). Therefore, efforts are being made to stimulate the patient's immune effector cells to recognize and destroy cancer cells. To this end, several different active immune therapies are currently under investigation, e.g. vaccination with whole cells (Sondak and Sosman, 2003), Trefzer et al., 2004), proteins, peptides (Otto et al., 2005, Slingluff et al., 2004), nucleic acids encoding the respective antigens (Gruenebach et al., 2005) or combinations thereof.

Carcinogenesis is a process depending on genetic and epigenetic alterations accumulating in transforming cells. Nevertheless, many steps necessary for tumor progression e.g. proliferation, invasion, angiogenesis, and metastasis are influenced by microenvironmental factors such as growth factors, angiogenic factors, cytokines, and proteolytic enzymes. During transformation, reciprocal interactions occur between neoplastic and adjacent normal cells, i.e. fibroblasts, endothelial, and immunocompetent cells. In general, stroma cells contribute 20-50% to the tumor mass, but the stromal compartment may account for up to 90% in several carcinomas. The microenvironment influences the stroma cells in such a way that they rather promote tumor progression than inhibit it by allowing vasculo- and angiogenesis, recruitment of reactive stromal fibroblasts, lymphoid and phagocytic infiltrates, secretion of peptide mediators, and proteolytic enzymes, as well as the production of a modified extracellular matrix (ECM). In contrast to cancer cells, tumor stroma cells are genetically more stable so that at least some immune evasion mechanisms of tumors do not apply for these cells. Nevertheless, stroma cells differ from their normal counterparts by upregulation or induction of various antigens. Some of the tumor stroma-associated antigens (TSAAs) are highly selective for the tumor microenvironment. It should be noted that some TSAAs may be expressed in the neoplastic cells as well and that they are not confined to one histiotype, indeed, they may be expressed by a broad spectrum of solid tumors. Thus therapies designed to target the tumor stroma are not restricted to a selected tumor entity (for a review see Hofmeister et al., 2006).

Since current therapies for most tumor entities are inefficient, there is still an unfulfilled need for pharmaceutical compositions for the treatment of solid tumors. The selection of the targeted antigen is essential for efficient treatment over a prolonged period of time without the development of therapy resistance. This is achieved by providing the embodiments characterized in the claims, and described further below.

### SUMMARY OF THE INVENTION

The present invention is directed to a nonamer peptide for the treatment of solid tumors, said peptide
is derived from fibroblast activation protein α (FAPα);
specifically binds to HLA with an affinity corresponding to an IC₅₀ value of less than about 50 μM in a competitive binding assay,
induces a T cell response in a subject,

wherein
position No. 2 of said nonamer peptide is leucine (L), isoleucine (I) or methionine (M), and position No. 9 of said nonamer peptide is leucine (L), valine (V) or isoleucine (I).

The present invention also concerns a nonamer peptide for the treatment of solid tumors, said peptide
is derived from fibroblast activation protein α (FAPα);
specifically binds to HLA with an affinity corresponding to an IC₅₀ value of less than about 50 μM in a competitive binding assay,
induces a T cell response in a subject,

wherein
position No. 2 of said nonamer peptide is leucine (L), valine (V), methionine (M) or proline (P); and/or
position No. 3 is aspartic acid (D), glutamic acid (E) or lysine (K); and/or
position No. 5 is lysine (K) or arginine (R); and/or position No. 9 is tyrosine (Y), lysine (K), phenylalanine (F), leucine (L), methionine (M), or isoleucine (I).

It is a further object of the present invention to provide a nonamer peptide for the treatment of solid tumors, said peptide
is derived from fibroblast activation protein α (FAPα);
specifically binds to HLA with an affinity corresponding to an IC₅₀ value of less than about 50 μM in a competitive binding assay,
induces a T cell response in a subject,

wherein
position No. 3 of said nonamer peptide is aspartic acid (D) or glutamic acid (E); and
position No. 9 of said nonamer peptide is tyrosine (Y).

Further, the invention provides a nonamer peptide for the treatment of solid tumors, said peptide
is derived from fibroblast activation protein α (FAPα);
specifically binds to HLA with an affinity corresponding to an IC₅₀ value of less than about 50 μM in a competitive binding assay,
induces a T cell response in a subject,

wherein
position No. 2 of said nonamer peptide is leucine (L), valine (V) or methionine (M), and
position No. 9 of said nonamer peptide is lysine (K), tyrosine (Y) or phenylalanine (F).

It is another object of the present invention to provide a nonamer peptide for the treatment of solid tumors, said peptide
is derived from fibroblast activation protein α (FAPα);
specifically binds to HLA with an affinity corresponding to an IC₅₀ value of less than about 50 μM in a competitive binding assay,
induces a T cell response in a subject,

wherein
position No. 2 of said nonamer peptide is proline (P), and
position No. 9 of said nonamer peptide is leucine (L) or phenylalanine (F).

The present invention also concerns a nonamer peptide for the treatment of solid tumors, said peptide
is derived from fibroblast activation protein α (FAPα);
specifically binds to HLA with an affinity corresponding to an IC₅₀ value of less than about 50 μM in a competitive binding assay,
induces a T cell response in a subject,

wherein
position No. 3 of said nonamer peptide is lysine (K),
position No. 5 of said nonamer peptide is lysine (K) or arginine (R), and
position No. 9 of said nonamer peptide is leucine (L).

Furthermore, the present invention relates to a nonamer peptide for the treatment of solid tumors, said peptide
is derived from fibroblast activation protein α (FAPα);
specifically binds to HLA with an affinity corresponding to an IC₅₀ value of less than about 50 μM in a competitive binding assay,
induces a T cell response in a subject,

wherein
position No. 2 of said nonamer peptide is proline (P), and
position No. 9 of said nonamer peptide is tyrosine (Y), phenylalanine (F), methionine (M), leucine (L) or isoleucine (I).

It is still another object of the present invention to provide a method for therapeutic or prophylactic treatment of a subject with a solid tumor, comprising the step of administering a composition with a nonamer peptide according to the invention.

### BRIEF DESCRIPTION OF THE DRAWINGS

FIG. 1 shows FAPα expression in normal skin and melanoma;

FIG. 2 shows the amino acid sequence of FAPα, HLA-A2-restricted human FAPα peptides are underlined and indicated in bold letters. At the bottom the HLA-A2 peptide binding motif is given.

FIG. 3 shows selected FAPα peptides and their binding to HLA-A2;

FIG. 4 shows the optimization of FAPα peptide epitopes;

FIG. 5 shows the detection of FAPα-specific T cells in PBMC (peripheral blood mononuclear cells)of melanoma patients by flow cytometry with HLA-A2/peptide multimers; FIG. 5A shows the results after in vitro stimulation with FAPα⁶³⁹⁻⁶⁴⁷ peptide while FIG. 5B shows the results after in vitro stimulation with FAPα mRNA transfected dendritic cells (DC);

FIG. 6 shows detection of FAPα-specific T cells by interferon-γ (IFN-γ) ELISPOT.

FIG. 7 shows the cytotoxic activity of peripheral blood lymphocytes (PBL) from melanoma patients to peptide loaded T2 target cells after stimulation with FAPα peptide-loaded DC—the bars represent different effector:target cell ratios.

FIG. 8 shows the peptide binding motifs of HLA-A1 and HLA-A3; and

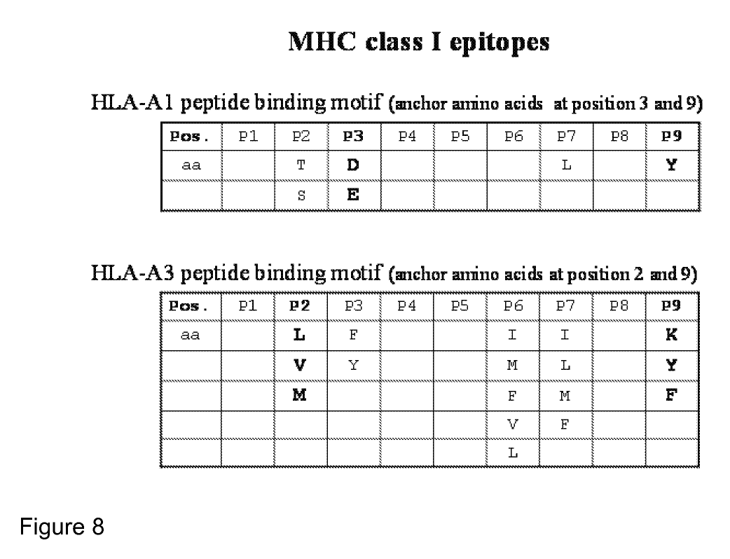

FIG. 9 shows the peptide binding motifs of HLA-B7, HLA-B8 and HLA-B35.

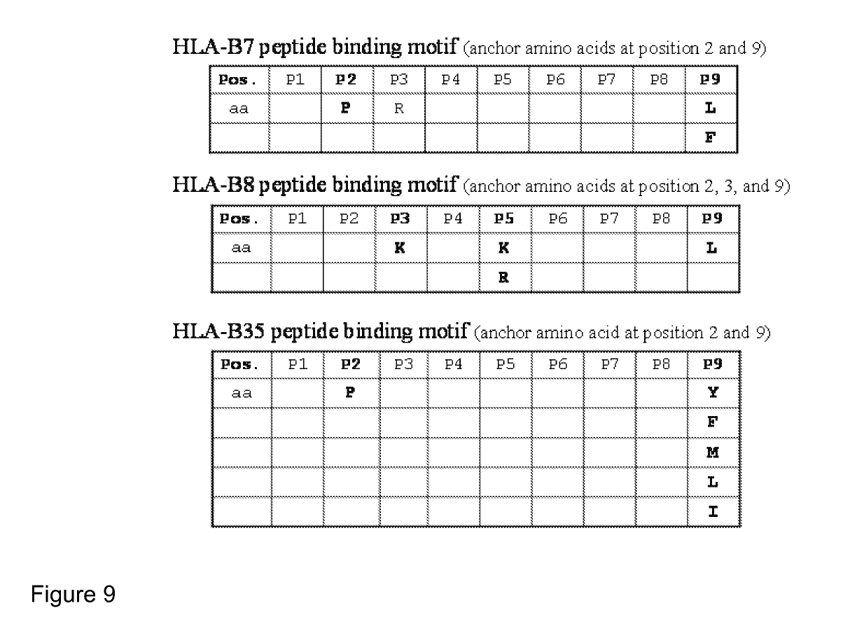

### DETAILED DESCRIPTION OF THE INVENTION

Immunotherapy has been widely investigated for its potential use in cancer therapy and it becomes more and more apparent that the selection of target antigens is essential for its efficacy. Indeed, limited clinical efficacy is partly due to immune evasion mechanisms of neoplastic cells, e.g. downregulation of expression or presentation of the respective antigens. Consequently, antigens contributing to tumor cell survival seem to be more suitable therapeutic targets. However, even such antigens may be subject to immune evasion due to impaired processing and cell surface expression. Since development and progression of tumors is not only dependent on cancer cells themselves but also on the active contribution of stromal cells, e.g. by secreting growth supporting factors, enzymes degrading the extracellular matrix or angiogenic factors, the tumor stroma may also serve as a target for immune intervention. To this end, several antigens have been identified which are induced or upregulated on tumor stroma cells. These tumor stroma-associated antigens (TSAAs) are characterized by an otherwise restricted expression pattern, particularly with respect to differentiated tissues, and they have been successfully targeted by passive and active immunotherapy in preclinical models. Moreover, some of these strategies have already been translated into clinical trials.

Therefore, the present invention relates to a nonamer peptide for the treatment of solid tumors, said peptide
is derived from fibroblast activation protein α (FAPα);
specifically binds to HLA with an affinity corresponding to an IC₅₀ value of less than about 50 μM in a competitive binding assay,
induces a T cell response in a subject,

wherein
position No. 2 of said nonamer peptide is leucine (L), isoleucine (I) or methionine (M), and position No. 9 of said nonamer peptide is leucine (L), valine (V) or isoleucine (I).

Fibroblast activation protein α (FAPα, seprase) is a cell surface protein with dual serine protease and dipeptidyl-peptidase activity. It is not expressed on normal adult tissue. However, FAPα expression is upregulated in the tumor micromilieu where it is mainly found on fibroblasts but can also be detected on tumor cells.

Although the role of FAPα in tumor progression is still controversely discussed, due to its strong induction in tumors FAPα is a promising target for cancer immunotherapy. By reverse immunology, the inventors have identified several HLA-A2 restricted peptides derived from FAPα that induce a human T cell response, measured by IFN-γ ELISPOT. Exchange of anchor amino acids of such peptides enhances their binding to HLA-A2 antigens thereby rendering them more immunogenic. Moreover, improved peptide/MHC affinity allowed the construction of recombinant HLA-A2/FAPα peptide complexes. Indeed, using such HLA-A2/FAPα multimers FAPα-specific T cells can be visualized in circulating blood of melanoma patients. The analysis of modified FAPα peptides in vivo using HLA-A2/kb transgenic mice demonstrated immunogenicity. This approach also serves to exclude major side effects of induced anti-FAPα immune responses in a preclinical setting. The peptides with the highest potential in inducing stroma-specific immune responses are then applied in immunotherapeutic studies in cancer patients.

The inventors' goal was to identify tumor stroma-associated antigens (TSAAs) that can be used to induce an immunological anti-tumor response. As a first step the inventors analyzed expression of fibroblast activation protein (FAPα, seprase) in normal skin, nevi, and melanoma by immunohistochemistry. FAPα was mainly expressed in cancer-associated fibroblasts (CAFs), but also by some nevus and melanoma cells. Subsequently, the inventors applied reverse immunology and tested peptide epitopes derived from this TSAA for their capacity to bind to MHC class I molecules. This analysis revealed seven FAPα with low to high binding affinity. Exchange of anchor amino acids in some low affinity peptides significantly enhanced their binding affinity. Functional tests (ELISPOT) with selected peptides allowed the in vitro induction of FAPα-peptide specific T cells in PBMC (peripheral blood mononuclear cells)of melanoma patients.

The identification of immunogenic peptides and their subsequent modification to improve their immunogenic potential is an invention in the field of life science. These peptides can be used for immune therapy of cancer. Carcinogenesis is a process depending on genetic and epigenetic alterations accumulating in transformed cells, allowing them uncontrolled proliferation, tissue invasion and finally metastasis. These alterations are accompanied by expression of tumor-associated antigens (TAAs), which are aberrantly expressed, mutated or newly induced proteins. Cells expressing TAAs can be recognized by cytotoxic T cells as these proteins in a cell are degraded and loaded onto MHC class I antigens (HLA class I antigens in humans) and presented on the cell surface to T lymphocytes. The recognition of MHC class I/peptide complexes by specific T cells via the T cell receptors (TCRs) can induce cytotoxic activity leading to apoptosis of the tumor cells. Immunotherapy targeting TAAs has been widely investigated for its potential use in cancer therapy. In general, up to date clinical trials tested vaccines directed against TAAs, which were highly cancer-type specific proteins. Moreover, the targeted proteins were not essential for the carcinogenic process. Hence, despite the fact that very specific immune responses were induced the clinical efficacy was very limited. As mentioned before, expression of most TAAs is not essential for tumor cell survival or its progression. Thus, limited clinical efficacy of these approaches is partly due to immune evasion mechanisms of neoplastic cells, e.g. downregulation of expression or presentation of the respective antigens. Moreover, this approach is tumor or tissue type specific, thereby limiting the number of patients that may be treated with a given vaccine. Without being bound by any scientific theory, the inventors believe that a possible solution to this problem relies on the fact that many steps in cancerogenesis e.g. proliferation, invasion, angiogenesis, and metastasis depend on microenvironmental factors such as growth factors, angiogenic factors, cytokines, and proteolytic enzymes supplied by stroma cells, e.g. fibroblasts, endothelial cells and macrophages. Furthermore, it has recently been proposed that cancer stem cells—i.e. cells which are essential for the maintenance of proliferative potential of the tumor and are difficult to attack by conventional and immunological means—critically depend on “survival” factors produced by tumor stroma cells.

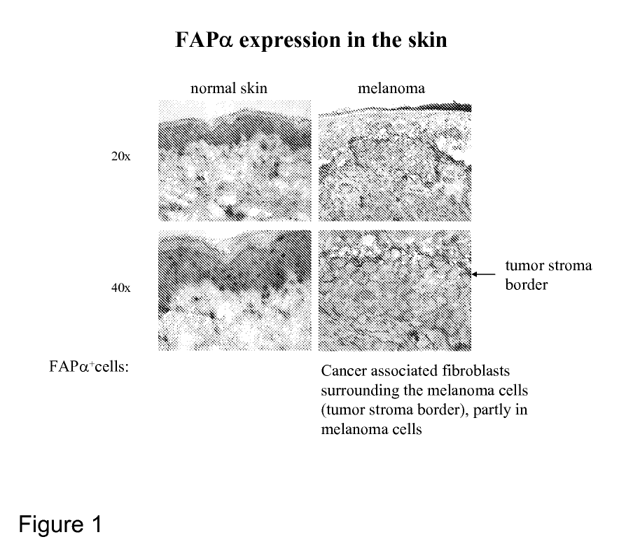

In order to induce effective cellular immune responses to tumor stroma cells and thereby destroying the tumor the inventors selected fibroblast activation protein a (FAPα, seprase) as immunotherapeutic target. FAPα possesses enzymatic activity and can degrade gelatin and process soluble factors in vitro. Its natural substrate has, however, not yet been identified. FAPα is selectively expressed on reactive stromal fibroblasts of a variety of solid tumors, whereas it is hardly present in adult normal tissue (FIG. 1). Indeed, FAPα is overexpressed in the stroma of more than 90% of common solid cancers and its overexpression is associated with enhanced tumor growth, invasion, angiogenesis, and metastasis.

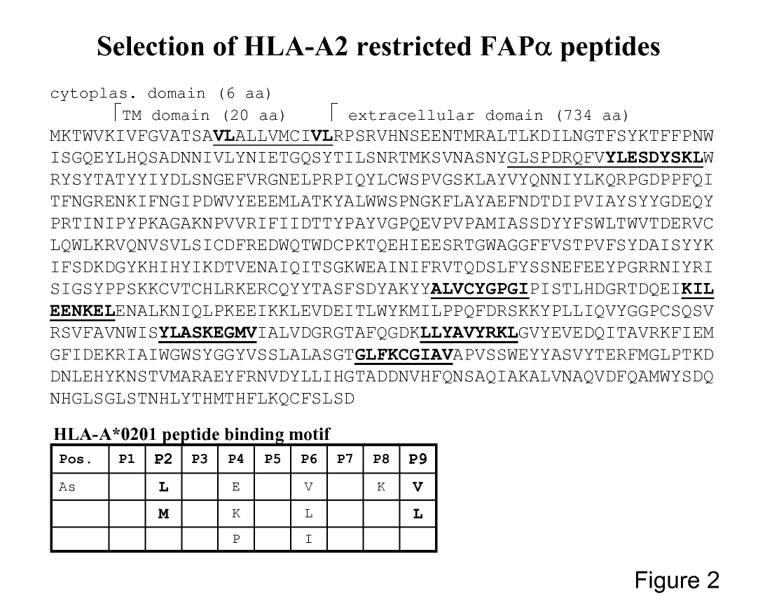

By “reverse immunology” the inventors identified FAPα peptides that bind to the common MHC class I antigen HLA-A2 and induce human T cell responses in vitro. Peptides presented by a given MHC class I molecule share a sequence motif corresponding to two or more essential amino acids (peptide anchor residues) in the context of a 9-10 amino acid long peptide. Such peptide binding motifs were used to predict potential HLA-A2-restricted peptide epitopes from FAPα (FIG. 2). Binding affinity of the selected peptides was controlled by competitive binding assay. This assay is based on the binding of the peptide to be tested and a fluorescein-labeled reference peptide to empty, acid-stripped HLA-A2-antigens. Reduction of the binding of the reference peptide by competitive binding of different concentrations of the tested peptide to the cells was analyzed by flow cytometry.

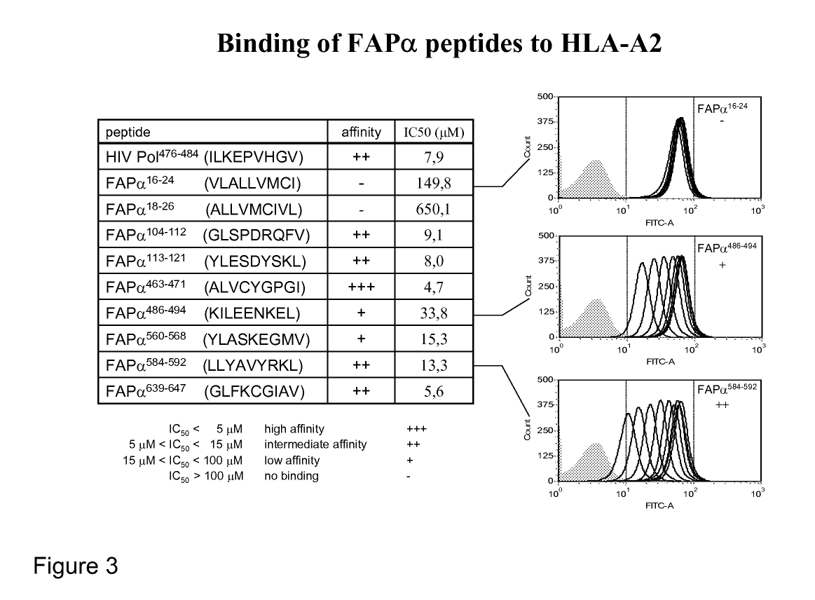

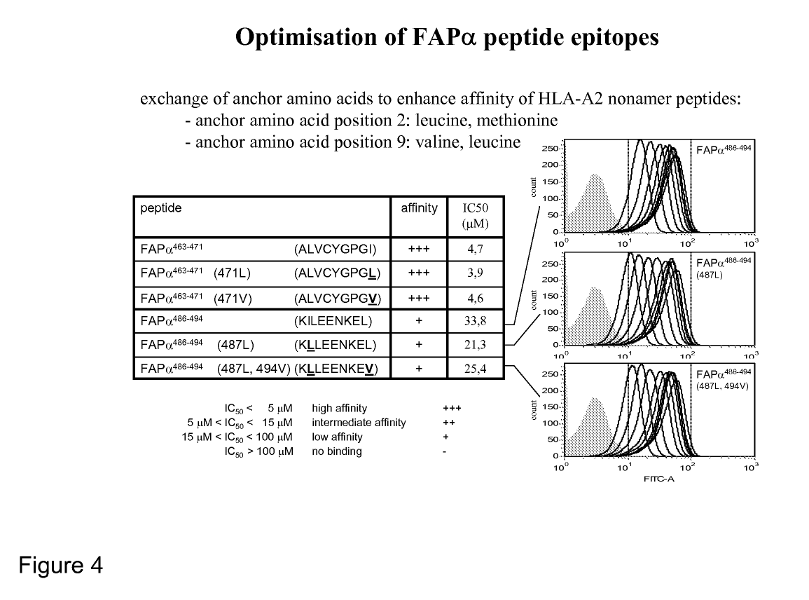

The inventors identified several FAPα-derived peptides with different degrees of affinity to HLA-A2 (FIG. 3). Amino acid substitutions at the anchor positions 2 (leucine, methionine) and 9 (leucine, valine) improved binding of selected peptides to HLA-A2 (FIG. 4). Importantly, the recognition of the peptides by T cells is not altered by this engineering.

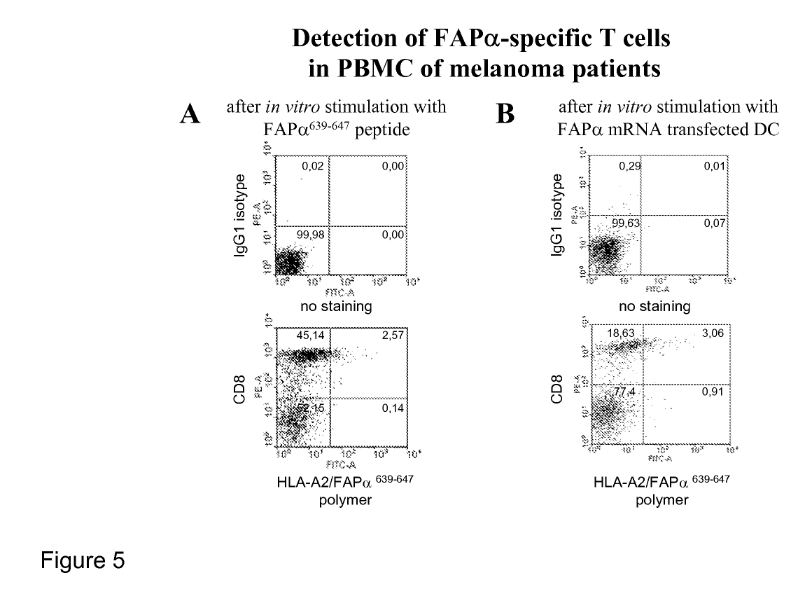

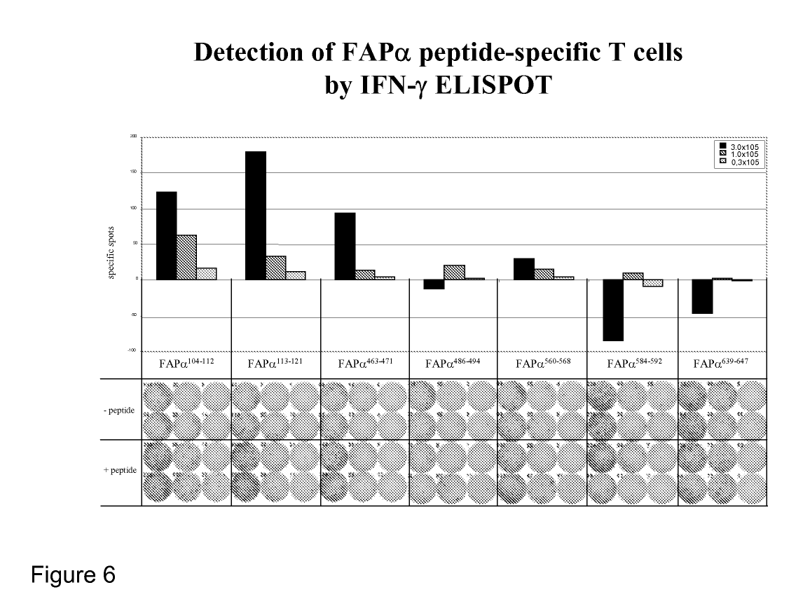

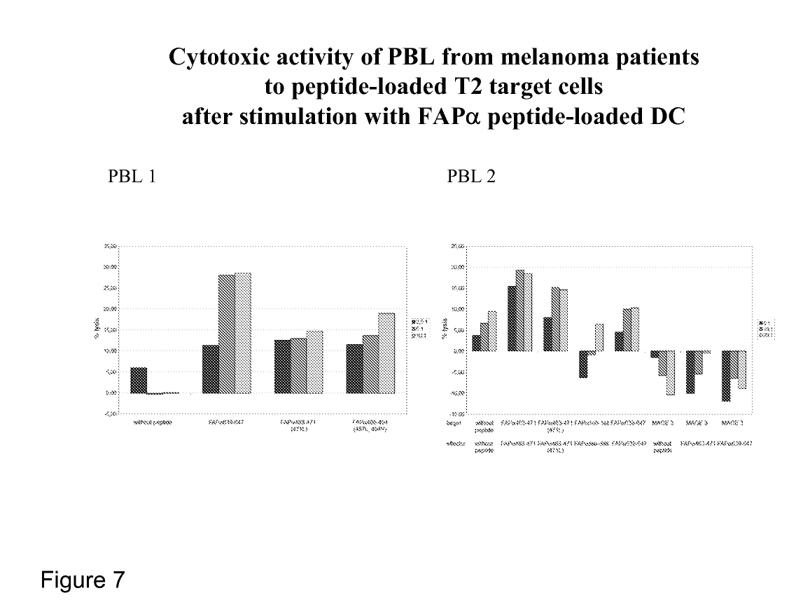

In the following the subsequent analysis is given in example for FAPα⁶³⁹⁻⁶⁴⁷. By means of HLA-A2/FAPα⁶³⁹⁻⁶⁴⁷ peptide polymers, consisting of HLA-A2/FAPα⁶³⁹⁻⁶⁴⁷ complexes and FITC-fluorophores the inventors were able to detect HLA-A2/FAPα⁶³⁹⁻⁶⁴⁷ specific T cells among PBMC of melanoma patients subsequent to in vitro stimulation with FAPα peptide-loaded dendritic cells (DCs) (FIG. 5A). In addition, HLA-A2/FAPα⁶³⁹⁻⁶⁴⁷ specific T cells can be detected after stimulation with DCs transfected with FAPα mRNA indicating that the FAPα⁶³⁹⁻⁶⁴⁷ epitope is generated by processing of endogenously expressed FAPα proteins (FIG. 5B). Functionally, IFN-γ ELISPOT assays, which measure the ability of T cells to respond to a certain peptide, demonstrate specific responses to FAPα peptides in PBMC of melanoma patients (FIG. 6). In addition, cyotoxic activity of PBMC stimulated with FAPα peptide loaded DC was directed to FAPα peptide loaded target cells whereas target cells without addition of peptide or loaded with an irrelevant HLA-A2 binding control peptide were not killed (FIG. 7).

Ongoing immunization studies with murine FAPα peptides in mice indicate that specific IFN-γ responses are induced by vaccination with no obvious side effects. Notably, FAPα knock-out mice do not demonstrate any phenotype and antibody targeted destruction of FAPα positive cells or immunization with FAPα-mRNA transfected DC only induced a delayed wound healing in accordingly treated mice. The occurrence of side effects, e.g. on wound healing or reproduction, after vaccination with FAPα peptides, however, will be further monitored in the murine system to exclude long term toxicity.

In summary, identification of FAPα peptides allows the development of a universally applicable vaccine since FAPα is expressed on cancer associated fibroblasts in a variety of cancers. Moreover, an effective therapy over a prolonged period of time should be possible as fibroblasts are genetically relatively stable reducing the risk of immune escape variants.

Thus, in a preferred embodiment, the present invention provides an inventive nonamer peptide, with the proviso:
if position No. 2 is leucine (L) than position No. 9 is leucine (L), valine (V) or isoleucine (I); and
if position No. 2 is isoleucine (I) than position No. 9 is leucine (L).

In alternative embodiments the nonamer peptide according to the invention bears the amino acid leucine (L) at position No. 2 and the amino acid isoleucine (I), leucine (L) or valine (V) at position No. 9.

In a further preferred embodiment the nonamer peptide according to the invention is a FAPα peptide, selected from the group consisting of

| | | |
|---|---|---|
| FAPα^{104–112} | (GLSPDRQFV, | SEQ ID NO: 4) |
| FAPα^{113–121} | (YLESDYSKL, | SEQ ID NO: 5) |
| FAPα^{463–471} | (ALVCYGPGI, | SEQ ID NO: 6) |
| FAPα^{486–494} | (KILEENKEL, | SEQ ID NO: 7) |
| FAPα^{560–568} | (YLASKEGMV, | SEQ ID NO: 8) |
| FAPα^{584–592} | (LLYAVYRKL, | SEQ ID NO: 9) |
| FAPα^{639–647} | (GLFKCGIAV, | SEQ ID NO: 10) |
| FAPα^{463–471} (471L) | (ALVCYGPGL, | SEQ ID NO: 11) |
| FAPα^{463–471} (471V) | (ALVCYGPGV, | SEQ ID NO: 12) |
| FAPα^{486–494} (487L) | (KLLEENKEL, | SEQ ID NO: 13) |
| and | | |
| FAPα^{486–494} (487L, | (KLLEENKEV. | SEQ ID NO: 14) |
| 494V) | | |

(See also FIGS. 3 and 4). Particularly preferred are the nonamer peptides described in SEQ ID NO: 6, 7, 9, 10, 11, 12 and 14.

In a particularly preferred embodiment, the HLA is HLA-A2.

Further, the invention provides a nonamer peptide for the treatment of solid tumors, said peptide
is derived from fibroblast activation protein α (FAPα);
specifically binds to HLA with an affinity corresponding to an IC₅₀ value of less than about 50 μM in a competitive binding assay,
induces a T cell response in a subject,

wherein
position No. 2 of said nonamer peptide is leucine (L), valine (V), methionine (M) or proline (P); and/or
position No. 3 is aspartic acid (D), glutamic acid (E) or lysine (K); and/or
position No. 5 is lysine (K) or arginine (R); and/or
position No. 9 is tyrosine (Y), lysine (K), phenylalanine (F), leucine (L), methionine (M), or isoleucine (I).

It is particularly preferred if HLA is selected from the group consisting of HLA-A1, HLA-A3, HLA-B7, HLA-B8, and HLA-B35.

In a further preferred embodiment, the invention provides a nonamer peptide for the treatment of solid tumors, said peptide
is derived from fibroblast activation protein α (FAPα);
specifically binds to HLA with an affinity corresponding to an IC₅₀ value of less than about 50 μM in a competitive binding assay,
induces a T cell response in a subject,

wherein
position No. 3 of said nonamer peptide is aspartic acid (D) or glutamic acid (E); and
position No. 9 of said nonamer peptide is tyrosine (Y).

It is particularly preferred if HLA is HLA-A1.

In a further preferred embodiment, the invention provides a nonamer peptide for the treatment of solid tumors, said peptide
is derived from fibroblast activation protein α (FAPα);
specifically binds to HLA with an affinity corresponding to an IC₅₀ value of less than about 50 μM in a competitive binding assay,
induces a T cell response in a subject,

wherein
position No. 2 of said nonamer peptide is leucine (L), valine (V) or methionine (M), and
position No. 9 of said nonamer peptide is lysine (K), tyrosine (Y) or phenylalanine (F).

It is particularly preferred if HLA is HLA-A3.

In a further preferred embodiment, the invention provides a nonamer peptide for the treatment of solid tumors, said peptide
is derived from fibroblast activation protein α (FAPα);
specifically binds to HLA with an affinity corresponding to an IC₅₀ value of less than about 50 μM in a competitive binding assay,
induces a T cell response in a subject,

wherein
position No. 2 of said nonamer peptide is proline (P), and
position No. 9 of said nonamer peptide is leucine (L) or phenylalanine (F).

It is particularly preferred if HLA is HLA-B7.

In a further preferred embodiment, the invention provides a nonamer peptide for the treatment of solid tumors, said peptide
is derived from fibroblast activation protein α (FAPα);
specifically binds to HLA with an affinity corresponding to an IC₅₀ value of less than about 50 μM in a competitive binding assay,
induces a T cell response in a subject,

wherein
position No. 3 of said nonamer peptide is lysine (K),
position No. 5 of said nonamer peptide is lysine (K) or arginine (R), and
position No. 9 of said nonamer peptide is leucine (L).

It is particularly preferred if HLA is HLA-B8.

In a further preferred embodiment, the invention provides a nonamer peptide for the treatment of solid tumors, said peptide
is derived from fibroblast activation protein α (FAPα);
specifically binds to HLA with an affinity corresponding to an IC₅₀ value of less than about 50 μM in a competitive binding assay,
induces a T cell response in a subject,

wherein
position No. 2 of said nonamer peptide is proline (P), and
position No. 9 of said nonamer peptide is tyrosine (Y), phenylalanine (F), methionine (M), leucine (L) or isoleucine (I).

It is particularly preferred if HLA is HLA-B35.

In a preferred embodiment, cancerous or precancerous lesion may be treated by FAPα nonamer peptides according to the invention, is selected from a group consisting of melanoma, basalioma, spinalioma, pancreas carcinoma, colon carcinoma, breast cancer and actinic keratosis.

The affinity of the nonamer peptide is decisive for the claimed invention. It is particularly preferred if the IC₅₀ value is about 5 μM to 50 μM, preferably 5 μM to 35 μM, further preferred 5 μM to 15 μM and most preferred less than 5 μM.

It is also preferred, if the subject to be treated is human.

The invention is further directed to a composition comprising a nonamer peptide according to the invention and a pharmaceutically acceptable carrier. Further pharmaceutical excipients, fillers and agents can be used, in order to formulate a composition which can be administered to a patient in need thereof.

Furthermore, the invention is directed to a method for a therapeutic or prophylactic treatment of the subject with a solid tumor, wherein the method comprises the step of administering a composition with a nonamer peptide according to the invention.

The appropriate concentration of the composition, in particular the nonamer peptide might be dependent on the particular peptide. The therapeutically effective dose has to be compared with the toxic concentrations; the clearance rate as well as the metabolic products play an important role as to solubility and formulation.

The therapeutic efficacy and toxicity of compounds can be determined by standard pharmaceutical procedures in cell cultures or experimental animals, e.g. ED₅₀ (the dose therapeutically effective in 50% of the population) and LD₅₀ (the dose lethal to 50% of the population). The dose ratio between therapeutic and toxic effects is the therapeutic index, and it can be expressed as the ratio LD₅₀/ED₅₀.

The above disclosure generally describes the present invention. A more complete understanding can be obtained by reference to the specific examples, which follow. They are provided for purposes of illustration only and are not intended to limit the scope of the invention.

### EXAMPLES

Material and Methods

Cells and Cell Lines

The human HLA-A*0201-positive cell lines T2, a TAP-deficient T cell leukemia/B cell line hybrid and JY, a B-LCL were cultured in RPMI 1640 supplemented with 10% heat inactivated FCS. After informed consent peripheral blood lymphocytes (PBLs) were collected from HLA-A2-positive patients with advanced malignant melanoma. PBLs from HLA-A2-positive healthy individuals were used as controls. PBLs were isolated using Lymphoprep separation (Axis-Shield PoC AS, Oslo, Norway) according to the manufacturer's instructions and used directly or frozen in FCS with 10% DMSO. PBL were cultured in RPMI/10% human AB serum. Dendritic cells (DCs) were generated from PBMC by adherence on culture dishes at 37° C. for 60 minutes in RPMI enriched with 10% human AB serum. Adherent monocytes were cultured in RPMI supplemented with 10% human AB serum in the presence of IL-4 (1000 U /ml) and GM-CSF (800 U/ml) for 6 days. DCs were matured by addition of IL1β (2 ng/ml), IL-6 (1000 U/ml), TNFα (10 ng/ml), and PGE2 (1 μg/ml). The next day the resulting mature DCs were pulsed with 10 μM peptide for 2 h at 37° C., irradiated (50 Gy) and 1×10⁵ DC/ml were used for stimulation of 1×10⁶ PBMC/ml in the presence of 40U/ml IL-2. IL-2 was added every 3-4 days.

Immunohistochemical Staining

Cryosections were stained with the FAPα-specific antibody F11-24 (Bender Med Systems GmbH, Vienna, Austria) and the Vector VIP or Vector Nova red system (Vector Laboratories, Burlingame, USA) according to the manufacturer's instructions.

Selection of FAPα Nonamer Peptides

Peptides derived from the full-length human FAPα protein were selected using both BIMAS (Parker et al. 1994) and SYFPEITHI (Rammensee et al. 1999) peptide binding algorithms available via the internet (http://bimas.cit.nih.gov/molbio/hla_bind/) and (http://www.syfpeithi.de/). Residues 2 and 9 of some peptides were changed to optimal anchor amino acids.

Competitive Binding Assay for Binding to HLA-A2 Molecules

The binding affinity of the synthetic peptides (GeneScript Corporation, Piscataway, N.J., USA) to HLA-A2 molecules was measured in the competitive binding assay as described previously (Kessler et al. 2003). The assay is based on the binding of the competitive peptide to be tested and the fluorescein-labeled reference peptide (FLPSDC(FI)FPSV, JPT Peptide Technologies GmbH, Berlin, Germany) to the acid-stripped HLA-A2 positive cell line JY. Reduction of the binding of the reference peptide is analyzed by flow cytometry. The percentage of binding inhibition of the FI-labeled reference peptide was calculated using the following formula:

(1−(MF_{reference+competitor peptide}−MF_{background})/(MF_{reference peptide}×MF_{background}))×100%

The binding affinities of the competitor peptides are expressed as IC₅₀ values, specifying their concentration sufficient for 50% inhibition of binding of the reference peptide. IC₅₀ was calculated by nonlinear regression analysis with software CurveExpert 1.3. Peptides with an IC₅₀≦5 μM were considered as high-affinity, with 5 μM<IC₅₀≦15 μM as intermediate-affinity, with 15 μM<IC₅₀≦100 μM as low-affinity, and with IC₅₀>100 μM as no binders.

Antigen Stimulation of PBL

To extend the sensitivity of the ELISPOT assay, PBL were stimulated once in vitro before analysis (McCutcheon et al. 1997). At day 0, PBL were thawed or freshly isolated from peripheral blood and plated in a cell concentration of 1×10⁶/ml and 2 ml/well in 24-well plates (Greiner GmbH, Frickenhausen, Germany) in X-vivo medium (Cambrex Biosciences, Verviers, Belgium) with 10% heat inactivated human AB serum in the presence of 10 μM peptide (GeneScript Corporation, Piscataway, N.J., USA). On day 1 and 4, 40 U IL-2/ml (Proleukin, Chiron GmbH, Munich, Germany) were added to the culture. The cultured cells were tested for reactivity in ELISPOT on day 7.

ELISPOT Assays

The IFN-γ ELISPOT assay was used to quantify peptide epitope-specific IFN-γ-releasing effector cells as described previously (Berke et al. 2000). Briefly, nitro-cellulose-bottomed 96-well plates (MultiScreen MAIP N45, Millipore GmbH, Schwalbach, Germany) were activated with 35% ethanol, washed with PBS and coated with anti-IFN-γ Ab (1-D1K, Mabtech, Hamburg, Germany). The wells were washed and blocked by X-vivo medium before adding 1×10⁴ stimulator T2 cells loaded with or without 10 μM peptide and 3×10⁵, 1×10⁵ or 3×10⁴ effector cells. After incubation overnight the wells were washed before addition of biotinylated secondary Ab (7-B6-1-Biotin, Mabtech, Hamburg, Germany). The plates were incubated for 2 h, washed, and streptavidin-enzyme conjugate (Streptavidin-ALP-PQ, Mabtech, Hamburg, Germany) was added. Incubation at room temperature for 1 h was followed by addition of enzyme substrate NBT/BCIP (Mabtech, Hamburg, Germany). The reaction was stopped by washing with tap water upon the appearance of dark purple spots. Spots were counted using the ImmunoSpot Series 2.0 Analyzer (CTL Cellular Technology Ltd., Schwabisch Gmuend, Germany). The peptide-specific CTL frequency was calculated from the numbers of spot-forming cells. All assays were performed in duplicates. Responders are defined as having an average number of >25 antigen-specific spots per 10⁵ cells (spots_{+peptide-}spots_{-peptide}).

Flow Cytometry and Antibodies

After peptide-specific stimulation with FAPα peptides or with FAPα mRNA transfected DC, generated as described previously (Fassnacht et al. 2005) PBLs were stained with HLA-A2/FAPα peptide FITC-labeled multimers (a kind gift of Jorgen Scholler, Dako, Glostrup, Denmark) in PBS/0.1% BSA/FCS for 30 min in the dark, followed by staining with anti-CD8-PE (Dako, Glostrup, Denmark) for 30 min at 4° C. in the dark. Samples were analyzed on BD FACS Canto (Becton Dickinson, Heidelberg, Germany) using WinMDi or FCS Express V3.

Cytotoxicity Assay

T2 target cells were labeled for 5 minutes with 1 μM and 0.05 μM CFSE (Molecular Probes, Invitrogen, Karlsruhe, Germany) in PBS and washed two times in PBS/10% FCS. Target cells labeled with the higher CFSE-concentration (CFSE-high) were incubated with 10 μM peptide for 2 h at 26° C. and shifted for 1 h to 37° C. CFSE^{high} and CFSE^{low} target cells were mixed 1:1 and 4×10⁴ cells were seeded in FACS tubes. After addition of effector cells in different concentrations the cells were coincubated for 4 h. Killing of target cells was analyzed by immunofluorescence on a BD FACS Canto (Becton Dickinson, Heidelberg, Germany). Reduction of the number of CFSE^{high} in comparison to CFSE^{low} cells indicates the specific lysis. Percentage of specific lysis was calculated as

[1−(% CFSE^{high}_{E+T}/% CFSE^{low}_{E+T})/(% CFSE^{high}_{T}/% CFSE^{low}_{T})]×100%.

E+T means sample with effector and target cells, whereas T indicates samples with target cells only.

### REFERENCES

Adami et al., (2003), Br. J. Cancer 89:1221-1227.
Berke et al., (2000), Leukemia 14:419-426.
Cho at al., (2003), Cancer Res 63:1555-1559.
Fassnacht et al., (2005), Clin Canc Res 11:5566-5571.
Gruenebach et al., (2005), Cancer Immunol Immunother 54:517-525.
Hofmeister et al., (2006), Cancer Immunol 55(5):485-494.
Kessler et al., (2003), Hum Immunol 64:245-255.
McCutcheon et al., (1997), J Immunol Methods 210:149-166.
Otto et al., (2005), Vaccine 23:884-889.
Parker et al., (1994), J Immunol 152:163-175.
Rammensee et al., (1999), Immunogenetics 50:213-219.
Slingluff et al., (2004), J Clin Oncol 22:4474-4485.
Sondak and Sosman (2003), Semin Cancer Biol 13:409-415.
Trefzer et al., (2004), Int J Cancer 110:730-470.
U.S. Cancer Statistics Working Group. United States Cancer Statistics (1991-2002 Incidence and Morality Web-based Report Version. Atlanta (Ga.): Department of Health and Human Services, Centers for Disease Control and Prevention, and National Cancer Institute; 2005. Available at www.cdc.gov/cancer/npcr/uscs. Accessed Jul. 17, 2006.

What is claimed is:

1. A nonamer peptide for the treatment of solid tumors, said peptide
is derived from fibroblast activation protein a (FAPa);
specifically binds to HLA with an affinity corresponding to an IC₅₀ value of less than about 50 μM in a competitive binding assay,
induces a T cell response in a subject,
wherein
position No. 2 of said nonamer peptide is leucine (L), isoleucine (I) or methionine (M), and position No. 9 of said nonamer peptide is leucine (L), valine (V) or isoleucine (I).

2. The nonamer peptide of claim 1, with the proviso:
if position No. 2 is leucine (L) than position No. 9 is leucine (L), valine (V) or isoleucine (I); and
if position No. 2 is isoleucine (I) than position No. 9 is leucine (L).

3. The nonamer peptide of claim 1, with the proviso:
if position No. 2 is leucine (L) than position No. 9 is isoleucine (I).

4. The nonamer peptide of claim 1, with the proviso:
if position No. 2 is leucine (L) than position No. 9 is leucine (L).

5. The nonamer peptide of claim 1, with the proviso:
if position No. 2 is leucine (L) than position No. 9 is valine (V).

6. The nonamer peptide of claim 1, wherein said nonamer peptide is FAPα peptide, is selected from the group consisting of

| | | |
|---|---|---|
| FAPα^{104–112} | (GLSPDRQFV, | SEQ ID NO: 4) |
| FAPα^{113–121} | (YLESDYSKL, | SEQ ID NO: 5) |
| FAPα^{463–471} | (ALVCYGPGI, | SEQ ID NO: 6) |
| FAPα^{486–494} | (KILEENKEL, | SEQ ID NO: 7) |
| FAPα^{560–568} | (YLASKEGMV, | SEQ ID NO: 8) |
| FAPα^{584–592} | (LLYAVYRKL, | SEQ ID NO: 9) |
| FAPα^{639–647} | (GLFKCGIAV, | SEQ ID NO: 10) |
| FAPα^{463–471} (471L) | (ALVCYGPGL, | SEQ ID NO: 11) |
| FAPα^{463–471} (471V) | (ALVCYGPGV, | SEQ ID NO: 12) |
| FAPα^{486–494} (487L) | (KLLEENKEL, | SEQ ID NO: 13) |
| and | | |
| FAPα^{486–494} (487L, | (KLLEENKEV. | SEQ ID NO: 14) |
| 494V) | | |

7. The nonamer peptide of claim 6, wherein said peptide is SEQ ID NO: 6, SEQ ID NO: 7, SEQ ID NO: 9, SEQ ID NO: 10, SEQ ID NO: 11, SEQ ID NO: 12 or SEQ ID NO: 14.

8. The nonamer peptide of claim 1, wherein said HLA is HLA-A2.

9. A nonamer peptide for the treatment of solid tumors, said peptide
is derived from fibroblast activation protein α (FAPα);
specifically binds to HLA with an affinity corresponding to an IC₅₀ value of less than about 50 μM in a competitive binding assay,
induces a T cell response in a subject,
wherein
position No. 2 of said nonamer peptide is leucine (L), valine (V), methionine (M) or proline (P); and/or
position No. 3 is aspartic acid (D), glutamic acid (E) or lysine (K); and/or
position No. 5 is lysine (K) or arginine (R); and/or
position No. 9 is tyrosine (Y), lysine (K), phenylalanine (F), leucine (L), methionine (M), or isoleucine (I).

10. The nonamer peptide of claim 9, wherein said HLA is selected from the group consisting of HLA-A1, HLA-A3, HLA-B7, HLA-B8, and HLA-B35.

11. A nonamer peptide for the treatment of solid tumors, said peptide
is derived from fibroblast activation protein α (FAPα);
specifically binds to HLA with an affinity corresponding to an IC₅₀ value of less than about 50 μM in a competitive binding assay,
induces a T cell response in a subject,
wherein
position No. 3 of said nonamer peptide is aspartic acid (D) or glutamic acid (E); and
position No. 9 of said nonamer peptide is tyrosine (Y).

12. The nonamer peptide of claim 11, wherein said HLA is HLA-A1.

13. A nonamer peptide for the treatment of solid tumors, said peptide
is derived from fibroblast activation protein α (FAPα);
specifically binds to HLA with an affinity corresponding to an IC₅₀ value of less than about 50 μM in a competitive binding assay,
induces a T cell response in a subject,
wherein
position No. 2 of said nonamer peptide is leucine (L), valine (V) or methionine (M), and
position No. 9 of said nonamer peptide is lysine (K), tyrosine (Y) or phenylalanine (F).

14. The nonamer peptide of claim 13, wherein said HLA is HLA-A3.

15. A nonamer peptide for the treatment of solid tumors, said peptide
is derived from fibroblast activation protein α (FAPα);
specifically binds to HLA with an affinity corresponding to an IC₅₀ value of less than about 50 μM in a competitive binding assay,
induces a T cell response in a subject,
wherein
position No. 2 of said nonamer peptide is proline (P), and position No. 9 of said nonamer peptide is leucine (L) or phenylalanine (F).

16. The nonamer peptide of claim 15, wherein said HLA is HLA-B7.

17. A nonamer peptide for the treatment of solid tumors, said peptide
is derived from fibroblast activation protein α (FAPα);
specifically binds to HLA with an affinity corresponding to an IC₅₀ value of less than about 50 μM in a competitive binding assay;
induces a T cell response in a subject,
wherein
position No. 3 of said nonamer peptide is lysine (K),
position No. 5 of said nonamer peptide is lysine (K) or arginine (R), and
position No. 9 of said nonamer peptide is leucine (L).

18. The nonamer peptide of claim 17, wherein said HLA is HLA-B8.

19. A nonamer peptide for the treatment of solid tumors, said peptide
is derived from fibroblast activation protein α (FAPα);
specifically binds to HLA with an affinity corresponding to an IC₅₀ value of less than about 50 μM in a competitive binding assay;
induces a T cell response in a subject,
wherein
position No. 2 of said nonamer peptide is proline (P), and
position No. 9 of said nonamer peptide is tyrosine (Y), phenylalanine (F), methionine (M), leucine (L) or isoleucine (I).

20. The nonamer peptide of claim 19, wherein said HLA is HLA-B35.

21. The nonamer peptide of claim 1, wherein said solid tumor is a cancerous or precancerous lesion.

22. The nonamer peptide of claim 21, wherein said cancerous or precancerouss lesion is selected from the group consisting of melanoma, basalioma, spinalioma, pancreas carcinoma, colon carcinoma, breast cancer and actinic keratosis.

23. The nonamer peptide of claim 1, wherein said IC₅₀ value is about 5 μM to 50 μM.

24. The nonamer peptide of claim 1, wherein said IC₅₀ value is about 5 μM to 35 μM.

25. The nonamer peptide of claim 1, wherein said IC₅₀ value is about 5 μM to 15 μM.

26. The nonamer peptide of claim 1, wherein said IC₅₀ value is less than 5 μM.

27. The nonamer peptide of claim 1, wherein said subject is human.

28. A composition comprising a nonamer peptide of claim 1 and a pharmaceutically acceptable carrier.

29. A method for therapeutic treatment of a subject with a solid tumor, comprising the step of administering a composition with a nonamer peptide of claim 1.

30. A method for prophylactic treatment of a subject who is susceptible to a solid tumor, comprising the step of administering a composition with a nonamer peptide of claim 1.


SEQUENCE LISTING (15 sequences)

SEQ ID NO: 1 - protein, 9 aa, Human immunodeficiency virus
ILKEPVHGV

SEQ ID NO: 2 - protein, 9 aa, Homo sapiens
VLALLVMCI

SEQ ID NO: 3 - protein, 9 aa, Homo sapiens
ALLVMCIVL

SEQ ID NO: 4 - protein, 9 aa, Homo sapiens
GLSPDRQFV

SEQ ID NO: 5 - protein, 9 aa, Homo sapiens
YLESDYSKL

SEQ ID NO: 6 - protein, 9 aa, Homo sapiens
ALVCYGPGI

SEQ ID NO: 7 - protein, 9 aa, Homo sapiens
KILEENKEL

SEQ ID NO: 8 - protein, 9 aa, Homo sapiens
YLASKEGMV

SEQ ID NO: 9 - protein, 9 aa, Homo sapiens
LLYAVYRKL

SEQ ID NO: 10 - protein, 9 aa, Homo sapiens
GLFKCGIAV

SEQ ID NO: 11 - protein, 9 aa, Homo sapiens
ALVCYGPGL

SEQ ID NO: 12 - protein, 9 aa, Homo sapiens
ALVCYGPGV

SEQ ID NO: 13 - protein, 9 aa, Homo sapiens
KLLEENKEL

SEQ ID NO: 14 - protein, 9 aa, Homo sapiens
KLLEENKEV

SEQ ID NO: 15 - protein, 760 aa, Homo sapiens
MKTWVKIVFGVATSAVLALLVMCIVLRPSRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPVVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEEYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSD